(19) United States
(12) Patent Application Publication
(10) Pub. No.: US 2010/0074963 A1
(43) Pub. Date: Mar. 25, 2010

(54) Red Palm Oil and Fish Oil Wound Dressing

(75) Inventor: Griscom Bettle, Sarasota, FL (US)

Correspondence Address: CHARLES J. PRESCOTT, P.A., SUITE 115, 2033 WOOD STREET, SARASOTA, FL, 34237, US

(73) Assignee: Biolife, L.L.C., Sarasota, FL (US)

(21) Appl. No.: 12/561,747

(22) Filed: Sep. 17, 2009

Related U.S. Application Data
(60) Provisional application No. 61/192,750, filed on Sep. 23, 2008.

(51) Int. Cl. A61K 8/97; A61K 8/98

(52) U.S. Cl. 424/523; 424/725

(57) ABSTRACT

A cosmeceutically acceptable formulation as a wound dressing including red palm oil and a triglyceride, preferably fish oil, preferably derived from salmon. Another aspect of this disclosure is providing a mixture of red palm oil and very long chain (>C_{>18}) omega 3 and 6 fatty acids. The formulation may also include one or more of the following: coconut oil and shea butter for anti-inflammatory activity and antimicrobial efficacy, fish collagen, an acidic preservative, a cationic surfactant, and various vitamins.

### CROSS-REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of the Invention

This invention relates generally to wound dressing and particularly to a red palm oil with fish oil dressing.

2. Description of Related Art

Lipids are indispensable to life. Lipids are triglycerides, which have three fatty acids bound to a glycerin backbone. Lipids come from vegetable sources such as olive, coconut, palm, borage, soybean, corn, flax and from animal sources such as beef, hogs, chicken, and emu and from marine sources such as cold water fish and the like.

The chemical structure of natural lipids varies widely. The length of the non-volatile fatty acid chain can vary from C₄ to >C₂₄. Traditional naming conventions are:

C₂₋₄ . . . volatile fatty acid (VFA)

C₈₋₁₂ . . . mid chain length triglyceride (MCT)

C₁₄₋₁₈ . . . long chain length triglyceride (LCT)

C_{>18} . . . very long chain length triglyceride (VLCT).

The degree of unsaturation in the chain can vary from zero to 6. The position of the last unsaturation site can vary with very different effects (ω3, ω6, ω9 where the number represents the carbon number, counting from the omega end (non carboxylic acid end) where the first double bond is encountered.). Finally, each of the three fatty acids can vary on a single glycerin backbone.

The chemical structure of synthetic lipids also varies widely. Mixtures of saturated and unsaturated fatty acids can be engineered to adjust melting points up and down to get cosmetically elegant triglycerides, whether formulated as emulsions, liquid lotions or as anhydrous semi-solid crèmes. Lipids are also chemically reactive both in a chemical reactor and naturally in the body. It is well known art to convert lipids into fatty alcohols, soaps or ethoxylates and the like in a chemical reactor. The body also uses lipids to make compounds necessary for good health. For example, lauric triglyceride, C₁₂, is converted into monolaurin wherein two of the fat legs are removed. Monolaurin, a glycerol fatty ester, is a natural antimicrobial compound. Similarly, caprylic acid, C₁₀, is part of the acid mantle of the skin. C₁₀ fatty acid is a natural antimicrobial compound. Palmitoleic acid, C_{16:1}, is another natural antimicrobial compound.

Omega 3 and omega 6 lipids are part of a lipid class called essential fatty acids. They are called essential fatty acids because the body cannot synthesize them and they must be ingested. The body has specialized enzymes which can increase or decrease the length of ω3 and ω6 fatty acids. The revised chain length is then converted enzymatically into a myriad number of biologically important compounds. However, the enzyme prefers C₁₈ omega 6 to C₁₈ omega 3, putting LCT omega 3 EFAs at a competitive disadvantage. If the omega 3 is already C_{>18}, then this enzymatic limitation is by-passed. The practical advantage of VLCT omega 3 versus LCT omega 3 is that the net concentration of C₂₀ omega 3 is increased when the enzyme-limiting step is by-passed. C₂₀ omega 3 is an anti-inflammatory precursor; C₂₀ omega 6 is an inflammatory precursor. Once a wound is no longer actively bleeding, anti-inflammatory compounds accelerate healing. For example, a phospholipid is a compound in which one of the three fatty acids in the triglyceride is replaced by a phosphorous-containing leg. Subsequent enzymatic manipulation forms a family of biologically active, hormone-like eicosanoids (C₂₀ compound) such as prostaglandins, thromboxanes and leukotrienes.

This chemistry is well known. The literature is full of references to the need for a diet rich in polyunsaturated fats, especially ω3 oils from fish. The chemistry of fats and fatty acids in the skin is also well known. Bodoprost et al. discloses that “human skin surface lipids consist of triglycerides (41%), wax mono esters (25%), free fatty acids (16%), squalene (12%) and other components (6%) such as cholesterol esters and cholesterol.” They go on to describe: “The fraction of free fatty acids of the stratum corneum represent a mixture of non-essential and essential fatty acids which are indispensable for the integrity of the epidermal barrier.”

Omega 3 fatty acids are well known as a dietary supplement to aid wound healing (McDaniel et al., 2008). Perversely, McDaniel found that the rate of healing was not improved by essential fatty acid ingestion, but the ω3 fats did affect one aspect of wound healing (“increased proinflammatory cytokine production at wound sites.”) Wound healing is a complex, multi-step process.

Kopas et al. (U.S. Pat. No. 7,531,196) discloses a “Cosmeceutical formulation containing palm oils”. In this specification, hereafter '196, an anhydrous combination of fractionated red palm oil with other vegetable oils, such as coconut oil, are disclosed as a substantially homogeneous, cosmeceutically acceptable topical formulation. A preferred embodiment is a room temperature crème in which the blended oil is still solid at 80° F. In another embodiment, the resulting formulation is preferably a lotion with a smooth homogeneous texture that has a melting point at or about body temperature. '196 discloses that: “In addition to the oils listed herein, it is understood that any additional oils high in EFAs may also be added to the claimed formulations.” Table 3 in '196 “shows the EFA profile for human skin lipids.” But Table 3 lists: “palmitic acid, Palmitoleic acid, stearic acid, oleic acid and linoleic acid”, 4 fatty acids that are not EFAs (i.e. not poly unsaturated ω3 and ω6 fatty acids; (linoleic is an ω6 C_{18:2} essential fatty acid)). '196 reinforces a lack of understanding of the definition of an EFA when he describes: “As shown in Table 3, these five (palmitic acid, Palmitoleic acid, stearic acid, oleic acid and linoleic acid) acids are part of the Essential Fatty Acids (EFAs) which form part of the lipid complex of the epidermis and aid skin barrier function by helping to control transepidermal water loss.”

'196 recognizes ω-3&6 as EFAs elsewhere in the specification. Without exception, the EFAs identified are of vegetable origin, such as listing flaxseed oil as a source of alpha-linolenic acid (a C_{18:3} omega-3 EFA). '196 states “In addition to the oils listed herein, it is understood that any additional oils high in EFA may also be added to the claimed formulations”. There is no mention of LCPUFAs (long chain (>C₁₈) polyunsaturated fatty acids) and no recognition of fish oil as a source of LCPUFA.

'196 claims anhydrous crèmes with a melting point <80° F. and recognizes that “Generally, the more unsaturated the fatty acid is, the more unstable it is”. Those skilled in the art also recognize that, the more unsaturated the fatty acid, the lower the melting point. To produce a crème with a high melting point would require limiting the quantity of unsaturation greater than 3. '196 recognizes this by stating: “The preferred EFAs to be added directly to the claimed formulation are alpha and/or gamma linolenic acid” (C_{18:3}), thus teaching away from fish oil that is low in C_{18:3} EFAs (typically 4.3% in salmon oil versus the 15% to 84% given in '196 as examples of high EFA oils) yet high in EFAs of C_{>18:>3} (typically 20.7% in salmon oil). Thus '196 teaches towards oils high in EFA and a high melting point. Skilled artisans will recognize that fish oil is high in EFA with unsaturation >3 and has a LOW melting point. Thus '196 teaches away from fish oil. '196 is correct in describing these Table 3 fatty acids as essential for controlling trans epidermal water loss, but is incorrect in describing them as essential fatty acids (acids which the body cannot produce itself).

'196 discloses that red palm oil is naturally high in Vitamin A precursors and Vitamin E such as various carotenoids (A) and tocopherols (E), stating: “Despite its beneficial effects, the carotenoids have not been successfully used in topical formulations because the pigments of these compounds produce severe skin discoloration.”

'196 claims that a “votated (agitated while cooling to supercool the fat) blend of palm oil” and other palm oil fractions . . . “is substantially homogeneous and discloses a means for producing an anhydrous, homogeneous semi-solid at room temperature, much like margarine. '196 does not recognize the effect of a room temperature liquid oil. In fact, it teaches away from room temperature liquid oils. One skilled in the art would recognize that a room temperature liquid fat mixture could have a higher percent of unsaturated fats than one which is solid at room temperature.

Crude palm oil also has a high concentration of squalene, a hydrocarbon with six unsaturated bonds and an important lipid in the skin. Crude palm oil is recovered commercially from the fruit and separately from the kernel. The fats in the fruit flesh are considerably different than the fats in the kernel. Red palm oil, from the flesh of the fruit, is a rich orange-red color. The color comes from the carotenoid compounds such as Vitamin A and Vitamin E. '196 teaches that the orange-red color is not desirable in the food and cosmetic industries and thus crude palm oil is refined.

Palm oil is approximately 50% saturated, 40% monounsaturated and 10% polyunsaturated. Palm (C₁₆) and oleic (C_{18:1}) are the predominate triglycerides. When palm oil is refined, it is separated into liquid and solid fractions, any free fatty acids are neutralized, and it is deodorized by passing hot oil over diatomaceous earth. Palm oil is a very large industry and there are myriad intermediate fractions, each with its own industrial description. For example, RBD oil is refined, bleached and deodorized; NBD oil is neutralized, bleached and deodorized; red palm olein is the liquid fraction, red palm stearin is the solid fraction.

Skin has an “acid mantle”, a very fine, slightly acidic (pH 4.5 to 5.5) aqueous film on the surface of the skin acting as a barrier to bacteria, viruses and other potential contaminants that might penetrate the skin. In wounded skin, the wound bed pH is neutral or slightly alkaline. The wound bed has lost its acid mantle protective film.

Fatty acids do not penetrate the stratum corneum easily, but liquid triglycerides, such as C₈, C₁₀ and unsaturated fats can. Solid triglycerides (>C₁₂) are not absorbed readily and form an occlusive layer over the skin. Once in the skin, natural processes convert the triglycerides into fatty acids, phospholipids and other chemicals. The saturated solid fats and free fatty acids stay on the skin surface and form an occlusive film to separate the skin from the external environment and to control transepidermal water loss (TEWL).

Omega 3 and omega 6 essential fatty acids are typically ingested fats because they have a characteristic odor that lingers on the skin and is cosmetically unacceptable. This odor is not masked by perfumes and fragrances. If the raw fat is not heated or exposed to oxygen, the rancid odor is minimized but not eliminated. Palm oil is naturally 50% saturated and 50% unsaturated but leaves a greasy feeling when applied to the skin. Coconut oil also leaves a greasy feeling. Surprisingly, mixing coconut oil, palm oil and fish oil does not leave a greasy feeling.

The nutrient and antioxidant value of palm oil and fish oil are well known. The traditional pathway is through the stomach and not through the skin. Coconut oil is well known as a topical treatment as well as an ingested treatment. It would be advantageous to deliver the nutrient and antioxidation benefits of palm and fish oil topically, particularly in mammals with chronic wounds. Topical application of fish oil emulsions is well known, but with mixed results. Jourdan et al. reviewed the EFA topical literature in 2005 and concluded: “In some cases, cutaneous application of linoleic (ω6 EFA) acid (from safflower/sunflower oil) may be beneficial although the literature is mixed.”

More recently, Faure et al., US2007/0128258, hereafter '258, disclosed an “oil-in-water emulsion gel comprising a hydrogel matrix, a gel-forming hydrophilic polymer covalently cross-linking a protein with a hydrophobic phase dispersed therein.” '258 describes “the resulting emulsion gel has a solid consistency and can undergo rapid swelling without discernible dissolution.” The '258 specification discloses that the omega3 oil-in-water wound dressing reduces the bacterial burden on wounds compared to olive oil (primarily C_{18:1}). '258 also discloses (claim 18) “a wound dressing comprising an omega-3 oil.” A careful reading of the specification equates “wound dressing” with “medical article” thus teaching away from anhydrous oil mixtures with omega-3 oil.

Borage oil is the highest (24%) ω3 EFA-containing oil from vegetables (gamma linolenic acid). Borage oil has been used as topical wound dressing for 1,500 years. Clearly one skilled in the art would know that borage oil is high in ω3 oil. Bettle et al., US20040122105, incorporated in its entirety and hereafter '105, discloses the use of borage oil in a topical, water-containing emulsion for treating wounds. The '258 specification does not define a wound dressing, but the FDA does in its 510k device regulations. There are three slightly different definitions, but common to all three is: a “ . . . device intended to cover a wound”. An ointment, solid, liquid or multi-phase dressing intended to cover a wound would be a wound dressing. Bettle '105 also teaches the use of CLA (conjugated linolenic acid), DHA and EPA in wound care emulsions. This teaching predates Faure '258 which teaches using “at least one of alpha-linolenic acid (CLA), eicosapentaenoic acid (EPA) and docosahexaenoic acid (DHA).

Faure '258 describes an elegant animal study in which pigs receive a 25 mm full thickness punch (an acute wound, not a chronic wound). Faure '258 concludes: “the omega-3 wound dressing (delivered to the wound) is able to modulate dermal regeneration, e.g. without over-stimulating of tissue regeneration, and prevent uncontrolled contraction of the wound.” The result is a healed acute wound without scarring. Faure '258 raises an important point in wound healing. The best healing is not necessarily the fastest. Over-stimulating growth of epithelial tissue may grow underlying structure that causes permanent scarring. A controlled rate of healing produces a better long-term resolution of a chronic or acute wound. Faure '258 describes oil-in-water emulsions of palm, coconut and fish oils for topical application on wounds, but does not disclose the use of red palm oil with fish oil.

### Wound Healing

It is important to understand how the body heals wounds, whether acute or chronic. Wound healing is complex and moves in distinct stages:
1. The first stage is the inflammatory stage. In this stage, compounds in the wound fluid attack necrotic tissue and break it down into component fractions that can be recycled to repair the wounds. This is a transitory stage in acute healing and a persistent stage in chronic wounds. In other words, this is a productive stage for a few days, but is non-productive if extended weeks, months or years.
2. The second stage is the proliferation stage or granulation stage. In this stage, temporary collagen structures are built as structure for vascular re-growth. The wound fills in from the bottom up; healing is vertical.
3. The third stage is the epithelialization stage. Epithelial cells grow from the wound margin, not the wound bed. In the epithelial stage, healing is horizontal.
4. The final stage of healing is the maturation or remodeling stage. The temporary collagen structures built in the proliferation stage are expedient structures which do not have the underlying strength of normal healthy skin. The body attacks these structures (after closure), intentionally destroys them and reconstructs the wound bed with stronger, denser structures. The first three stages might take several weeks to accomplish in a healthy wound; the maturation stage can take up to two years.

If the proliferation stage is accelerated, non-uniform tissue is created which leads to long term scarring. An ideal wound healing strategy is to let the wound heal at a natural rate. If the epithelialization stage is accelerated, the wound can close over a structure that is not strong enough. The wound may open again.

The body has many systems in which chemical signaling takes place. Bleeding control is an example with over a dozen choreographed steps (the clotting cascade) which leads to hemostasis. Wound healing also has signaling mechanisms which control when to inflame the wound, when not to, and when to start destroying the temporary structure built up to close a wound. It is no surprise then that McDaniel et al. would say in 2008: “Polyunsaturated fatty acids alter cytokine (a proinflammatory compound) production, but how this phenomenon specifically influences wound healing is not clearly understood.”

### Wound Dressing Characteristics

An ideal wound dressing would be one in which:
1. Excess exudate is absorbed, but the wound stays moist.
2. Wound bed pH is ˜5.5 to inhibit matrix metalloproteases (MMPs are inflammatory compounds designed to destroy collagen).
3. Fats and FFAs are available to insure that a robust acid mantle is maintained.
4. The wound margin is protected from excess moisture to inhibit maceration of the periwound.
5. The periwound away from the wound margin should be moisturized.
6. Vitamins and other minor nutrients are readily available.
7. The product should have strong antioxidant capacity to control peroxidase attack.
8. Inflammation is encouraged when appropriate, but discouraged when not.
9. Infection is brought under control such that microbial colonization does not inhibit healing.
10. Collagen is bio-available in the wound bed.
11. Cross-linking cations are available to strengthen collagen as it is formed during the proliferation stage.
12. Signaling precursors are bio-available such that the body can send the appropriate signal at the appropriate time.
13. Moisture should be allowed to escape during final wound closing.

Fortunately, essential fats (fatty acids), especially C_{20:ω3&6} PUFAs are the building blocks for many of the body's signaling mechanisms. Prior art embodiments use anhydrous refined fats or neutralized oil-in-water emulsions for skin care. Both strategies are fundamentally flawed. The refined fats specifically eliminate some chain lengths, not knowing whether some combination of these long chain lengths are used for important signaling purposes. Neutralized oil-in-water emulsions are used to fill a wound bed with water (40 to 75%) and functional ingredients (60 to 25%) of the emulsion. Any fatty acid is neutralized into soap. Thus the loading of the functional ingredients, limited by the volume of the wound bed plus the absorption of the covering dressing, is reduced because so much of the volume is taken up by water in the wound cavity.

Wound healing is complex and takes place in the wound bed and at the wound margin. But this assumes that the wound is contracting and healing. Venous ulcers, particularly, can double in size overnight. This typically occurs as a result of free radical attack by fatty peroxidases and similar compounds. Attention to wound healing also has to recognize the role that surrounding tissue plays.

An ideal wound dressing is one which moves the wound out of the inflammation stage and continuously and progressively moves the wound through the granulation and epithelialization stages while minimizing tissue that needs subsequent remodeling. Faure '258 describes the omega 3 effect on acute wounds versus omega 9 (oleic acid) on wounds that were not inflamed or infected.

### BRIEF SUMMARY OF THE INVENTION

This disclosure is directed to the novel composition of red palm oil and fish oil. When applied topically to mammalian skin, this mixture strengthens the acid mantle and reduces inflammation of the skin. Fish oil is high in very long chain omega 3 & 6 fatty acids but low in C₁₈ omega 3 & 6 fatty acid. The novel mixture can also be described as a mixture of red palm oil and very long chain (>C₁₈) omega 3 & 6 fatty acids.

A novel anhydrous wound dressing of red palm oil and fish oil such that the oil mixture is a substantially homogeneous liquid at ambient temperature is disclosed. When this mixture is subsequently mixed with coconut oil, the mixture has indirect antimicrobial properties when applied topically to skin. When virgin red palm oil, fish oil, coconut oil and shea butter are mixed together, the oil has anti-inflammatory activity, indirect antimicrobial efficacy, and moisturizing properties even though the mixture itself is substantially water-free. When red palm oil and fish oil are mixed with an acidic preservative, the acid mantle is strengthened further.

The term “fish oil” refers to oil extracted from fish carcasses. However, fish oil can come from algae (the start of the food chain which converts sunlight and CO₂ into fish oil, body mass and oxygen), small vertebrate fish like sardines (or menhaden) and large predator fish like salmon. The term “fish oil mixture” as used herein refers to a preferred embodiment thereof including fish oil and red palm oil. There are other optional ingredients. Fish oil itself is defined as predominantly a triglyceride wherein the weight ratio of C_{>18} PUFA/C_{≦18} PUFA>1. For example, wild caught Sockeye Salmon oil (See Table 2) has 10.0% C₁₈ PUFA and 28.2% C_{>18} PUFA. The ratio is 28.2/10.0=2.82 (which is >1.0). Borage oil is a highly unsaturated vegetable oil (C_{18:2}=39%; C_{18:3}=24%) which has only trace amounts of C_{>18} PUFA. Therefore, the ratio is zero. Flax seed oil has 58% C_{18:3} and 14% C_{18:2} or a total of 72% C₁₈ PUFA. Therefore, the ratio is zero.

Surprisingly, it is possible to formulate a product that meets the requirements of an ideal wound care dressing. But the product is not a single device but rather multiple devices in fluid communication which together form an in situ healing system. An anhydrous organic mixture preferably of virgin coconut oil, virgin red palm oil, minimally processed fresh sockeye salmon oil and traditionally-recovered shea nut oil such that the mole ratio of C₁₆ to C₁₈ is 1.0, the mole ratio of the sum of C_{4 to 10}+monounsaturated oils to the sum of all polyunsaturated oil is 2.0, and that this oil is a free-flowing non-winterized fluid at <80° F., hereafter called a “fish oil” mixture. This fish oil mixture is spread onto the wound bed, the wound margin and rubbed into the skin surrounding the wound. For example, on a patient with a venous ulcer on the shin, the fish oil mixture would be rubbed into the entire leg from the knee to the toes and everywhere in between. The fish oil mixture is optionally enriched with 1% fish collagen, and Vitamins not found in vegetable oil, like Vitamin K. The fish oil mixture can also be preserved with a preservative system to have antimicrobial properties.

The fish oil mixture has an odor characteristic of fish and an orange-red pumpkin color when first rubbed in. About five minutes after rub-in, the color is gone and the odor is mostly gone. About ten minutes after rub-in, the odor is functionally gone. The skin feel is soft and luxurious. Some of the oil rubs in immediately; some is absorbed in 5 to 10 minutes; some is not absorbed and stays on the surface. The absorbed fats transport the colored molecules and the fish-odor molecules into the skin. Non-winterized oil has two phases. It is preferably packed in a single use blister to insure a constant, reproducible dose is delivered.

If the fish oil mixture is optionally enriched with a cationic surfactant and winterized at 20° C., then the malodor and color is gone in 3 minutes. An optional mixture is a mixture of red palm oil and fish oil alone. The liquid fats help transport the colorful compounds through the stratum corneum. When winterized, this oil is best used post healing to prevent future breakdown. By winterizing this oil, the skin does not feel greasy. This oil is preferably packed in a multi-use, oxygen barrier pump bottle.

On wounds which are deep, the fish oil mixture alone may be insufficient. A second embodiment is made from the previously-described fish oil (about 50%) and a mixture of fish collagen, divalent cations, Vitamins A, C, D, K & E, other antioxidants and various herbs. The anhydrous mixture is such that the fish oil mixture is adsorbed onto the powder materials to make a paste which is thick enough to stick on a cotton-tipped swab. This paste, referred to hereafter as collagen paste, can be packed into tunnels and under crevices. The paste embodiment is preferably packed in small jars with metal lids. The fish oil mixture covers the entire treatment area and the collagen paste is packed into the wound bed space up to about the level of the surrounding skin. It is well known that the collagen absorbs exudate and forms an egg-shell, occlusive crust over the wound bed. But surprisingly, as the moisture is absorbed, the adsorbed oil is released. Thus there is a continuing, timed release of oil and free fatty acids to the wound bed. This oil is optionally winterized to prevent forming a too-strong occlusive film over the wound bed.

The red palm oil and fish oil mixture can also be made into an aqueous emulsion for long-term skin maintenance after a wound is healed. People who form a first venous ulcer are prone to form a second and a third because the underlying venous insufficiency causes skin breakdown. A low concentration of red palm oil and fish oil in a traditional oil-in-water or water-in-oil emulsion, as taught in '105, can help prevent future breakdowns by making the skin healthy.

### Mechanisms of Action

1. Minimally processed fats have a small percentage of free fatty acids (FFA).
2. Two free fatty acids are produced when a single triglyceride is hydrolyzed; the remaining moiety is a glycerol monoester such as glycerol monolaurate when a C₁₂ triglyceride breaks down. [Glycerol monolaurate is a well-known antimicrobial compound.]
3. Fats shorter than C₁₂ are liquids at room temperature; fats ≧C₁₈ are solids at body temperature; monounsaturated and polyunsaturated fats are liquids at room temperature. Liquid fats penetrate the stratum corneum faster than solid fats.
4. When the red palm oil/fish oil mixture is rubbed into the skin, the polyunsaturated fats enter the skin first. The semi-liquid fats penetrate somewhat later and appear to drag the colorful vitamins into the skin.
   a. When red palm oil, fish oil, coconut oil and Shea butter are mixed together such that there are approximately equal moles of C_{8&10}) omega 6 and omega 3 fats, then the absorption rate (i.e. disappearance of visual color) is accelerated.
5. The solid fats coat the skin and are not absorbed; winterization changes the thickness of this occlusive film. The FFA is not absorbed, and tends to buffer the acid mantle at pH˜5.5. This strengthens the acid mantle, particularly in wound beds where there are no remaining hair follicles. [Hair follicles and sweat glands, working together, are the natural producers of the fatty acids in the acid mantle.]
6. The omega 3 fatty acids are converted into prostaglandin 1 & 3, the anti-inflammatory prostaglandins; the omega 6 is converted into prostaglandin 2, the pro-inflammatory prostaglandin. High prostaglandin 2 would account for the inflammation effect described by McDaniel et al. The pro-inflammation effect would also be seen with the omega 6 linoleic acid described in the '196 patent.
7. The omega 3 fatty acids are well known anti-inflammatory compounds; when they are in the dermis, they assist in draining fluid from the dermis into the various vascular and lymph systems. Clinically, less exudate is observed because it flowed into the body not out from the body.
8. When the acid mantle is strengthened and the pH is maintained at ˜5.5, then biofilms are less likely to form; the wound does not reach critical colonization and is not infected.
9. When the wound is not infected, the bioburden is reduced and there is less necrotic tissue to debride. With less time spent debriding necrotic tissue and slough, the doctor is more productive, costs are lower and patient comfort is increased.
10. When a wound is closing, the surface dries out. Winterized oils have higher TEWL and the surface dries, allowing the wound to close and form tough, durable outer skin.

### Red Palm Oil

Kopas '196 describes the use of red palm oil fractions in an anhydrous semi-solid mixture with the optional use of coconut oil and oils high in essential fatty acids, describes EFAs incorrectly as saturated and monounsaturated fats from palm oil. '196 states: “the presence of high amounts of EFA in palm oil has the benefit of reducing trans epidermal water loss and preventing dry skin.” EFAs described in Table 3 of Kopas '196 are not essential fatty acids defined as “fatty acids that cannot be constructed within an organism (humans) from other components by any known pathways and therefore must be obtained by diet . . . . There are two families of EFAs, ω3 and ω6.” '196 describes the fatty acid composition of palm oil fractions. Only one fatty acid in palm oil is essential, linolenic C_{18:3}) and that is only present at 0.37% (in crude palm oil), not the “presence of high amounts of EFA in palm oil” taught by '196. Clearly when Kopas '196 discusses EFAs, he refers to a range of important fatty acids, but not essential fatty acids as understood by the world at large.

Red palm oil is obtained from the endocarp of the palm fruit (the soft flesh); palm oil is obtained from the seed (palm kernel oil). The oils are very much different. Red palm oil has the highest level of antioxidants of any seed crop. The carotenoids and tocotrienols et al. give red palm oil its distinctive color. These highly colored compounds are not readily absorbed by the skin and stain the skin surface and any clothing or bedding surface it comes in contact with. Fish oil mixture is characterized by an odor which many sense as “rancid”. These two issues have precluded the use of fish oil and red palm oil in topical cosmetics or medicaments.

Red palm oil is a broad mixture of fatty acid chain lengths. Some of the longer fats freeze at room temperature (˜20° C.). These fats settle slowly, a process called winterization. However, some of these room-temperature solid fats are needed to form an occlusive layer over the skin to reduce TEWL.

### Fish Oil

Surprisingly, mixing red palm oil and fish oil, particularly from wild caught Alaska salmon, resolves both issues. The fish oil helps transport the colorful compounds into the epidermis, where it no longer is available to stain; the solid fats coat the skin after the fish oil has penetrated and minimize any residual odor. Those skilled in the art will find particular ratios that work most effectively, but in general a weight ratio of red palm to fish oil close to 1:1 is ideal. Once these issues are resolved, mixing oils and other ingredients can make remarkably unique products whether anhydrous or as water emulsions.

In general, red palm oil provides a high level of antioxidants; the very long chain omega 3 polyunsaturated fatty acids (VLPUFA) are well known as anti-inflammatory compounds. Natural fats break down during separation with hot water. Some of the fats are broken down into free fatty acids (FFA). Together, the wide range of fats and the residual FFAs rejuvenate the acid mantle. This is particularly important in sick people who develop non-healing wounds such as venous ulcers, diabetic foot ulcers, pressure ulcers and the like.

Winterization is a slow process with an amorphous separation between clarified supernatant and solid fat. It would be advantageous if the separation was sharp such that the fraction of liquid fats entrained in the solid fraction was minimized. Surprisingly, the addition of cationic surfactants, such as benzethonium chloride, to the anhydrous mixture tends to concentrate the solid fraction and exclude entrained liquid fats. The decanted liquid is saturated with the solid fat, transmits light and rubs in easily. The solid fraction is firm and easily separated.

Surprisingly, after all these years of people using oils for cooking, eating and skincare, there is a mixture of virgin coconut, virgin red palm, salmon oil and shea butter fat that is a liquid at room temperature and is rapidly absorbed. In addition, this mixture transports the unsaturated, odiferous fats and the colorful carotenoids through the stratum corneum where the fats are no longer smelled and the color is no longer seen.

### Polyunsaturated Fatty Acids (PUFA)

The PUFA are transported through the stratum corneum rapidly. The mono unsaturated fats and the shortest saturated fats are slower to be absorbed. It is not understood how, but apparently the transport of the color bodies is easier when some fats precede the color bodies. The longer chain fats are not readily absorbed and fill in the imperfections in the skin to make a smooth, triglyceride-filled surface. The uniform mostly-saturated oil film on the surface acts as a moisture and odor barrier for moisture ingress and egress and to keep off-odors contained. The effect is to keep internal moisture in the skin (i.e. moisturize the skin) and to keep environmental moisture out of the skin (e.g. to prevent maceration of the wound margin by exudate). Once the unsaturated fats are absorbed and covered by saturated fats, the strong odor characteristic of VLPUFA is not smelled. The C₁₂ is only partially absorbed, but is converted by the body into monolaurin (glycerol monolaurate) in situ. The C_{16:1}, from the salmon oil, is absorbed and becomes a natural antimicrobial compound. The C₁₀ is absorbed and becomes a natural antimicrobial part of the acid mantle. The eclectic mix of not-absorbed fats has a hand-feel that is substantially non greasy and cosmetically acceptable after winterizing and decanting. Shea butter is the primary contributor of C₁₈ fat.

### Carotenoids

The carotenoids and the ω3 fats change the skin chemistry. The carotenoids are powerful antioxidants and inhibit some of the well-known peroxidase chemistry. The ω3's are primarily C_{20:X} eicosanoid precursors and a fundamental building block of skin chemistry.

### Natural Antimicrobials

When the acid mantle is reinvigorated by the natural antimicrobials and the acid-based cationic preservative system, infection in the wound is reduced. Chronic wounds move out of the inflammation stage, where they have been stuck, into the granulation and proliferation stages and on to epithelialization and closing, just like acute wounds. Closing is accelerated with winterized/decanted mixtures.

### Omega 3

Omega 3 fats are well known as anti-inflammatory compounds when ingested and can be anti-inflammatory when applied topically at particular ratios with other fats. This disclosure is better understood from the following Examples.

It is an object of this disclosure to achieve the chemical precursor attributes of true essential fatty acids, the carotenoid chemistry of red palm oil, and the indirect antimicrobial effect of coconut oil could be combined in a mixture in which the odor and skin discoloration issues are mitigated so that use on wounds is acceptable to everyday customers.

Another object is to dress a wound with an anhydrous product that was anti-inflammatory, controlled infection, modulated the effect of peroxidase free radicals and provide collagen structure for ordered rebuilding of the wound site such that scarring was minimal and that the ultimate closure was complete and durable.

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference to the drawings and by study of the following descriptions.

### DETAILED DESCRIPTION

### Ratios of Fish Oil

When the mole ratio of C₁₆=C_{18:1}=1 (Ratio 1) and the sum of C₄₋₁₀+monounsaturated fats divided by the sum of the poly unsaturated fats (PUFA) equals 2 (Ratio 2), the resulting mixture has little odor and is completely absorbed by the skin in about 5 minutes without staining and without residual odor on the skin.

These two ratios define a unique space for these four oils:
Coconut oil is primarily C₈₋₁₂ with a small amount of C_{18:1}, and is a solid at room temperature.
Red palm oil is primarily a mixture of C₁₆ and C_{18:1} and C_{18:2}, and is a solid at room temperature.
Salmon oil is a mixture of C₁₄ to C₂₄, much of it as VLPUFA, and is a liquid at room temperature.
Shea butter or shea nut oil is primarily a mixture of C₁₈ and C_{18:1}, and is a solid at room temperature.

C_{18:1} is common to all four fats and is also in both ratios. This ties the interactivity of the mix to the contributing fats to achieve these ratios.

Ratio 1 is used to describe the mix, split into absorbable fats and not-readily absorbable fats. The longer saturated fats form an occlusive seal over the skin which keeps moisture in. The short chain saturated and mid to long chain unsaturated fats are liquid at room temperature and pass through the stratum corneum into the subsurface skin.

Ratio 2 describes the importance of the mole ratio of short chain saturated and mono unsaturated fats to total PUFA. Three of the constituent fats are solid at room temperature. The salmon oil lowers the melting point of the mixture such that the mixture is a fluid at room temperature. The mixture has a characteristic fish odor and is the color of salmon eggs as applied topically to skin.

Most of the oil is absorbed readily, but there is strong odor and strong color on the surface. About 5 minutes later, there is only a slight odor and no color. After 10 elapsed minutes, there is almost no detectable odor and no color. The surface of the skin has a slight greasy feel such as the feel when cosmetically elegant moisturizing emulsions are rubbed into the skin.

When the oil mixture is winterized for 7 days at 20° C., the color and odor disappears faster, in about three minutes. Winterization also standardizes the concentration of long chain saturated fats. When the solids are removed, the concentration of long chain saturated fats in the supernatant is the saturation concentration. The saturation concentration does not leave a greasy feeling on the skin surface.

### Example 1

Shea butter (7.8 g), organic virgin coconut oil (22.3 g), organic virgin red palm crude oil (34.6 g) and wild caught sockeye salmon (33.6 g) were melted together with indirect heat. Fish collagen (1 g) was added along with Vitamin K1 (0.7 g as 5% K1 on sugar and acacia gum). The mixture was stirred in an anaerobic environment. This admixture is referred to as “fish oil mixture” and was packed into 1.5 ml aluminum/plastic oxygen-protective single serve packets.

### Example 2

The mixture (51 g) of Example 1 was mixed with a sieved, dry combination of Fish Collagen (38 g), allantoin (0.5 g), MSM (1 g), Aloe Vera 200x powder (0.3 g), copper gluconate (0.15 g), ascorbic acid (2 g), calendula powder (0.75 g), grape seed extract (0.75 g), zinc monomethionine (0.75 g), curcumin powder (0.75 g), Vitamin D3 (0.7 g of a 25% powder), Vitamin K1 (0.7 g). Added to the fish oil is Vitamin E (0.7 g of a 50% oil), lycopene (0.7 g of a 6% oil), Pro Vitamin A (0.7 g of a 20% oil) and Geogard 361 (0.6 g as a preservative). The mixture was stirred until uniform and then packed in 0.5 g/packet, inert-gas blanketed SARAN single serve packets.

### Example 3

Dan is a 70 year old man who lives alone with four dogs. His lifestyle is not conducive to wound healing. Dan presented with an infected venous ulcer on his right shin that had been open for over two years. Dan was treated for two months with compression bandages and other best practices. His physician finally recommended hospitalizing Dan for sharp debridement (surgically enlarging the wound to get to tissue that is uninfected and apt to heal).

Dan refused this course of treatment and instead was treated with the mixture of Example 2 which was rubbed into Dan's entire lower leg weekly; the wound bed was packed with collagen paste weekly. New paste was placed over any residual without removing the old dressing. The leg was wrapped with a best practices compression bandage.

After twelve weeks, Dan's wound was resolved completely. The skin around the wound sloughed off in great hunks. When the wound resolved, Dan's skin around the wound was pink, hydrated and healthy.

### Analysis of Example 3

Dan's wound was colonized with pathogens which kept the wound in the inflammation stage. The oil mixture entered the skin and was converted into fatty acids, fatty esters and C₂₀ compounds which resolved the infection, allowing the wound to move into the proliferation phase. The collagen paste absorbed the excess exudate but still kept the wound bed moist. The wound moved out of the inflammation stage and into the proliferation stage. Normal healing then began.

### Example 4

George presented with a venous ulcer on the top of his right foot and severe swelling of the lower extremities. Four inches of bone were exposed as a result of a motorcycle accident and post surgical infection. George was treated at a local lymphedema center using specialized massage therapy and short-stretch compression bandages. The wound was stuck in the inflammation stage.

Fish oil mixture was applied to the entire lower leg. Collagen paste was packed into the open wound. After 24 hours the swelling was completely gone. After 6 months, the wound was resolved and George was released by the clinic.

### Analysis of Example 4

This venous ulcer was deep, so collagen paste was used. The fish oil mixture was absorbed by the skin and biologically converted into antimicrobial compounds which eliminated the infection which was the root cause of the swelling. Once the infection was resolved, the lower leg could drain and healing could begin naturally.

### Example 5

Cheryl, a young, heavy-set restaurant manager who was on her feet all day presented with two venous ulcers on the front of either leg. She said the wounds had doubled in size since the day before.

Fish oil was applied to both legs. An overwhelming foul, rotted fish odor was produced in seconds after applying the fish oil mixture. Cheryl said she would try the fish oil mixture only until her boyfriend returned from a trip in 3 days because the odor was so bad.

By day 3, the odor was tolerable and the wound started to heal. Further treatment with fish oil mixture was refused.

### Analysis of Example 5

Cheryl's wound was growing because of free radical attack at the wound margin. The foul odor came from direct oxidation of the unsaturated sites in the fish oil mixture, turning essential fats into an oxidized by-product. The antioxidants in the fish oil mixture quenched the free radicals and allowed the wound to begin healing naturally.

### Example 6

Mr. C had a venous ulcer on the inside of one ankle. As the ankle ulcer resolved, Mr. C developed a diabetic foot ulcer on his big toe. Both were treated with fish oil mixture and collagen paste and bandaged with compression bandages about halfway up the calf. After 12 weeks, both wounds were resolved, but above the compression bandage a new set of three venous ulcers appeared. Fish oil mixture was applied from the knee to the toe for three weeks. All ulcers were resolved. Fish oil mixture was applied from the knee to the toe prophylactically. No more ulcers presented themselves.

### Analysis of Example 6

Mr. C has a chronic venous insufficiency which puts him at risk for venous ulcers. The fish oil mixture and collagen paste resolved the pre-existing ulcers. The new breakdown began outside the treatment area. By treating the entire leg prophylactically, further breakdowns were avoided.

### Example 7

The fish oil mixture of Example 1 was allowed to sit unmixed at room temperature (˜72° F.) for several weeks. A loose cloudy precipitate formed which, when shaken, increased the opacity of the fluid. The cloudy fluid poured like olive oil and rubbed in easily and had very little odor. The Example 1 fluid was packed in single dose packages.

### Analysis of Example 7

The Example 1 mixture contains C₁₆, C₁₈ and C₂₀ triglycerides. These fats are solids at room temperature. Over time, these fat fractions precipitate, but do not form hard solids. The solids are entrained in the liquid fats and flow out of the package completely. By using single dose packages, the consumer always gets a uniform dose. The suspended solid fats rub onto the skin surface easily, creating an occlusive film over the skin.

### Example 8

Miss C is a 90 year old, fair-skinned woman with chronic arterial deficiency in the lower leg. After two operations to improve blood flow (primarily increasing flow in the carotid artery), Miss C refused further surgery and opted for palliative care. Her legs hurt all the time and she had extensive stasis dermatitis over the entire lower leg. Fish oil mixture was rubbed gently onto the entire lower leg daily for two weeks. The dead skin absorbed the yellow fraction of the fish oil color and flaked off in great sheets. Beneath the exfoliated dead skin, the skin was pink and healthy. Miss C had no more leg pain after two weeks.

### Analysis of Example 8

The fish oil mixture partially penetrated Miss C's skin and reduced inflammation in the leg. This did not improve blood flow into the leg, but did improve blood flow out of the leg. With less fluid build-up in the leg the pain was reduced. The dead skin absorbed some of the fish oil mixture and lubricated it. The dead skin was released (exfoliated). The underlying skin was healthier because the oils stimulated skin chemistry and restored the skin to its prior health.

### Example 9

Mr. X presented with bi-lateral lymphedema in both lower legs. Mr. X had fish oil mixture rubbed into the left leg every three days for four weeks. The right leg did not have oil. Both legs were wrapped with short-web compression using best practices. After 2 weeks, the fish oil mixture leg was visibly better than the non-oil leg.

### Analysis of Example 9

The fish oil mixture reduced inflammation and allowed the lymph system to drain naturally.

### Example 10

The mixture of Example 1 was reproduced except without collagen added. A level study of collagen-added was performed from 0% to 1%. The solubility of fish collagen was determined to be 0.33%.

### Example 11

An oil mixture of shea butter (7%), virgin coconut oil (23%), red palm oil (34.5%), sockeye salmon oil (35%), 0.2% Vitamin E (50% Vitamin E in palm oil) and 0.4% Geogard 361 (a commercial preservative with phenoxyethanol, dehydroacetic acid, salicylic acid, benzoic alcohol, benzethonium chloride and benzyl alcohol) was gently mixed and heated just enough to melt the fats.

The pH was 3 as oil and 5 when diluted with distilled water. Geogard 361 in water has a pH of 3. The mixture was winterized at 20° C. for 5 days. The supernatant was decanted; the solids were discarded.

When the supernatant was rubbed into the skin, the skin was initially discolored; after 3 minutes, there was no visible skin color change (from the original) and there was only the slightest fish odor.

### Analysis of Example 11

The pH of the oil is the pH of the Geogard. Natural oils have a small fraction of free fatty acids. When the oil is mixed with water, the free fatty acids (pKₐ at ˜5.5) buffer the solution to about the pH of the acid mantle (5.5). There was no residual greasy feeling.

### Example 12

The mixture of Example 11 was rubbed into the periwound, the lower leg and wound bed of patients presenting with venous ulcers. After many patients and many months, the Principal Investigator (PI) reported that there was an overall reduction in bio-burden such that there was a significant reduction in the re-sterilization of disposable sharp debridement tools. The PI also reported a significant reduction in stasis dermatitis. There were no odor complaints from patients. The PI was able to increase his patient throughput by 20%. The skin was colored after the first rub-in, but then three minutes later, the color was absorbed.

### Analysis of Example 12

The oil mixture of Example 11 provided free fatty acid to the skin such that the acid mantle was reinforced. A robust acid mantle, in conjunction with the preservative, reduced the skin pH and kept the skin from critical colonization, reducing the bio-burden. When a wound is infected, excess exudate is formed along with biofilm slough. This slough is typically removed via sharp debridement. When the biofilm was reduced, the need for sharp debridement was reduced. Fewer instruments were sterilized and the Doctor's personal productivity increased because he did not have to spend time debriding. As a result, he could increase his patient count.

There were no odor complaints because benzethonium chloride, a cationic surfactant, forms an ion pair with free fatty acid and releases a proton and a chloride. This reaction takes place in the acid mantle over the skin at body temperature. The ion pair joins with two other ion pairs and forms a hydrogen bond with water (a hydronium ion) to form a “Yen (¥)-shaped” acid hydrate with a positive charge in the middle (see Bettle et al. '105). The negative charge in the skin attracts the acid hydrate to the skin surface. The acid hydrate partially penetrates the stratum corneum and partially does not. This opens the SC to more rapid penetration by large molecules such as vitamins in the red palm oil and fish oil. The colorful molecules are transported into the skin and are no longer seen after three minutes.

Unsaturated fatty acid esters have a strong characteristic odor (“old fish odor”). The fatty acid esters are mixed with saturated fats as applied. As the unsaturated fats are absorbed, they leave behind a smooth uniform layer of saturated fat over the rapidly-disappearing unsaturated fats. The saturated fats cap the remaining unsaturated fats and malodors are kept under the saturated fat layer and thus are not perceived.

### Example 13

Emulsions as described in '105 were modified to incorporate red palm oil and fish oil. The emulsions were used daily on patients who had previous venous ulcers but which were currently healed. After 180 days of use, out of 100 patients, <1% of patients developed new venous ulcers. With the control, without the red palm oil and fish oil, the incidence of recurrence was 5%.

### Example 14

The oil with red palm oil, fish oil, coconut oil and Shea nut oil was mixed with 0.13% benzalkonium chloride. This red color was absorbed very rapidly.

### Analysis of Example 14

As with Example 12, benzalkonium chloride is a reactive quaternary surfactant and forms an ion pair with the free fatty acid. Three ion pairs form an acid hydrate with a hydronium ion. The positively-charged acid hydrate is attracted to the negatively-charged skin surface and opens the barrier. Vitamins are transported rapidly into the skin.

### Example 15

The fish oil was replaced with a mixture of vegetable oils such that the Example 11 mixture and the Example 15 mixture had equal moles of omega 6 & omega 3. Example 15 had no LCPUFAs. Both products were tested on a broad cross section of patients. The Example 11 results were significantly better.

### Analysis of Example 15

The body uses a common enzyme to lengthen PUFAs. Omega 6 C₁₈ fatty acids out-compete C₁₈ omega 3s for this enzyme. Example 11 started with longer PUFAs (C_{>18}), so the omega 3s were not at a disadvantage. Example 11 was a stronger anti-inflammatory mixture.

### Example 16

To the Experiment 1 mixture, the Vitamin K was removed and Vitamins E and A were added. Winterization was conducted at 20° C. The color was considerably darker because of the Vitamin A. But surprisingly, the winterization was more efficient, with sediment of just 7.5% V/V.

### Example 17

Roger had a chronic wound on his right elbow. Non-winterized fish oil was applied daily for two weeks. The wound resolved in 4 days, but did not form a strong, durable outer skin. Fish oil mixture was used every other day and the wound healed completely in four more days.

Patty used a non-winterized and then a winterized oil on a surgically-removed skin cancer site. The wound partially closed and then healing stalled with the non winterized oil. With the winterized oil, the wound healed completely without scarring.

### Analysis of Example 17

Winterization reduces the thickness of the residual occlusive film. The absorbed fats and nutrients cause the wound to granulate and epithelialize. But the surface needs to dry out to form tough, durable outer skin. Winterizing allows the surface to dry out faster than with the non-winterized oil. The decanted solid residue fraction is incorporated in the oil used to moisturize the periwound. The super-dry skin associated with stasis dermatitis benefits from the extra, saturated-not-absorbed fat.

### Example 18

Daphne is a 67 year old Caucasian female who presented with a broken blood vessel in the left eye. The entire eye was purple. Fish oil mixture was applied and the purple color went away overnight.

### Analysis of Example 18

The VLPUFA omega 3 in the fish oil mixture is an anti-inflammatory compound. When applied to the eye, the edema drained from the eye, removing the discoloration.

### Example 19

The Example 1 collagen/fish oil paste was reapplied to the wound and immediately covered with a cut-to-fit-shape piece of dry collagen matrix. Examples of suitable collagen matrix materials are J&J Fibracol, Prisma and Promogran. In this example, Fibracol was used because it has the highest collagen level and is also the lowest cost matrix.

Fibracol is a dry woven mesh of >80% collagen. The wound specialist measures the wound and cuts a “jigsaw piece” of matrix to fit the open area. The wound is packed with collagen paste and before the fats melt and run out, the matrix is pressed into place. The wound is wrapped for 7 days.

### Explanation of Example 19

Chronic wounds typically have considerable depth. Dressings are placed in and around the wound bed. In Example 19, the wound bed was completely filled with paste, providing more paste than can be quickly absorbed and a distal insulating layer of paste over any proximal melt. The collagen matrix is placed over the paste and is wet by the paste, creating a sandwich effect between the wound bed, the paste and the matrix. Molten fats are absorbed by the matrix, wetting the matrix, and do not run out. The wound and dressing are then wrapped with a pressure-offloading means for 7 days. The excess wound dressing is collagen-rich and is absorbed by the wound bed over 7 days. Because it is absorbed, it is not removed at the next visit and the fragile wound bed is not disturbed. The wound heals more rapidly because the granule buds are not touched.

### Example 20

Collagen paste is added to the wound bed and optionally matrix is added as in Example 19. In this wound, the wound is infected with a biofilm, traditionally called slime or slough. Historically, slough is sharply debrided out of the wound as a way to eliminate topical infection. An unwanted by-product of sharp debridement is disruption of the granulating wound bed. The wound healed rapidly and slough was not debrided but went away automatically.

### Explanation of Example 20

A biofilm is a complex structure in which over 99% of bacteria worldwide live. Colonizing bacteria settle on a surface and bond themselves to the surface with strong adhesive combined with multi-valent cations such as calcium, magnesium and iron. Synergistic bacteria, including pathogens, form complex structures in which they are protected from distal sanitizers and blood-borne systemic antiseptics. Nutrients come in through the distal side of the biofilm, thus keeping systemic proximal antiseptics from entering the biofilm. Distal topical antiseptics kill bacteria on the surface but do not penetrate to the middle of the biofilm and thus the biofilm survives.

Biofilm disruption by external chelators is well known. The mechanism postulated is two-fold: disruption of the cell wall by divalent cation extraction (which weakens a bacterium) and binding of structure-building divalent cations (which weakens the biofilm structure).

The collagen paste mechanism of action is the same with distinct differences. The acid mantle of the skin is on the proximal side of the biofilm and it is weakened in a chronic wound because there are no hair follicles (one of the co-producers of fatty acid). Sweat glands, the other co-producer of fatty acid do produce fatty acids (primarily C₁₈ fatty acid from ingested animal fat and vegetable fat). The acid mantle reduces the pH to 5.5, a pH that inhibits growth, but is not lethal. Thus the biofilm slough can grow in a normal wound.

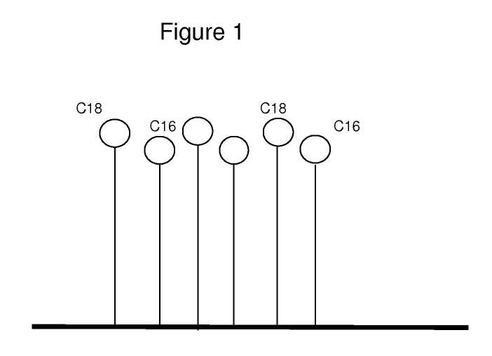

When shea butter, palm oil and fish oil are mixed, there is a resulting mixture of oils plus a small amount of free fatty acids. The fish oil helps transport the oils (C₁₆ and C₁₈ triglyceride) through the biofilm. [As a food source, the biofilm lets it into its matrix.] The skin gradually absorbs residual fat and rejects free fatty acid. The carboxylic head of C₁₈ and C₁₆ fatty acids are identical; the length of the tail is different. It is well established that mixing equi-mole concentrations of surfactants with different tail lengths can increase the packing density as shown in FIG. 1. In fact, there is a 1.707 increase in packing density in the acid mantle (1+sin 60°=1.707) when there is an equi-mole mixture of C₁₆ and C₁₈ fatty acids. Fatty acids are well known “builders”, compounds which bind Ca⁺⁺. [C₁₈ fatty acid is the builder in Tide Liquid detergent.]

Bacteria are shaped somewhat like a drum. The cell wall strength is obtained by divalent cations such as Ca⁺⁺. Without wishing to be bound, it is hypothesized that the increased packing density of fatty acids on the proximal side (i.e. the unprotected side) of the biofilm is enough to extract Ca⁺⁺ from the cell wall and from the biofilm structure to weaken the bacterium and ultimately dislodge the biofilm. The slough is absorbed and the wound is uninfected. Support for this hypothesis comes from emerging research wherein 30% ethanol and 4% citrate (another well-known builder) are effective antiseptics. The alcohol swells the bacterium and the citrate binds the cell wall calcium.

Banin et al “Chelator-Induced Dispersal and Killing of P aeruginosa Cells in a Biofilm” showed that EDTA can kill gram positive biofilms. Davis, US20080317815, teaches that some fatty acids can cause a physiological dispersion response in a biofilm.

Thus the novel mixture of red palm oil and fish oil and optionally shea nut butter in a chronic wound prevents formation of biofilm and infection therefrom and the wound heals faster by either killing or dispersing the biofilm. The chelator comes from the proximal surface, not the distal surface, thus attacking the binding of the biofilm to the wound.

### Example 21

The collagen paste of Example 19 and the matrix are used to pack the wound bed. A water-emulsion of various nutrients, the tertiary amide of Bettle '105, and red palm oil and fish oil is mixed into a crème using well-known techniques. This crème is applied to the periwound and wound margin of a venous ulcer. The wound granulates and closes rapidly without infection. The periwound skin color is pink and healthy appearing. Leg swelling is reduced.

### Explanation of Example 21

The Example 20 wound bed granulation occurs as described. The water emulsion of Example 21 is rapidly absorbed into the skin because of the tertiary amide, allowing fish oil and red palm oil to pass into the dermis. The fish oil reduces inflammation in the periwound, increasing leg drainage and reducing swelling. The C_{8&10} fats are converted into fatty acids in the dermis and reduce the pH in the lower leg. The Bohr Effect (hemoglobin oxygen is dropped off at slightly lower blood pH) adds extra oxygen to the circulation-impaired lower leg, simulating an increase in circulation even though there is no increase in net flow. The periwound skin is pink and healthy. The C₁₈ fats are not transported into the dermis and create an occlusive layer over the lower leg and importantly the wound margin. The wound margin is not macerated and epithelialization proceeds rapidly. The wound closes.

### Fish Oil Emulsions Protocol for Leg and Foot Care

Fish oil mixtures are used in anhydrous and water-based emulsions for skincare and wound healing, particularly chronic wound healing. Of particular interest are fish oil, red palm oil and collagen mixtures. These mixtures transport some collagen into the dermis along with well-known natural vitamins such as A and E. These mixtures work well as described but with deficiencies, particularly in terms of the size of the dose. Size of the dose is important because it is typical to treat wounds weekly and then cover the wound with a dressing and then off load the wound so there is no direct pressure on the wound or the periwound. A larger dose provides sufficient nutrients to continuously feed the wound over a seven-day period. Table I lists the details of ingredients of the preferred embodiment of a foot and leg crème.

### Venous Ulcer & Diabetic Foot Ulcer Protocol Weekly Visits

A protocol change to avoid melting and runout of this crème of Table I resolved this issue. The revised method is:
1. Debride the wound as necessary
2. Transfer the collagen paste with +2% lidocaine USP onto your index finger
3. With a tongue depressor or equivalent, fill the wound cavity.
   a. The patient's body heat will melt the fat in the paste (coconut and palm oil) and distribute nutrients throughout the wound bed.
   b. The exudate will be absorbed by the fish collagen (40% fish collagen by weight); the adsorbed oils (salmon oil, red palm oil, coconut oil and shea butter) will be released into the wound bed and absorbed by the wound over the course of a week's time; the fish collagen will form an egg-shell-like crust with the exudate. The mixed length of free fatty acid will create a robust acid mantle.
4. Cut a piece of Fibracol collagen matrix to fit the wound (or use more-expensive/less % collagen Prisma or Promogran).
5. Apply the Fibracol over the collagen paste and lightly tamp into place
   a. The Fibracol will absorb any not-yet-absorbed oil and help prevent messiness.
   b. The paste will wet the Fibracol which helps it eventually to be absorbed by the wound bed.
6. Squeeze a palmful of Foot & Leg crème into your hand; rub into the leg from the knee to the tip of the toe until absorbed.
   a. Most of the time this is done to both legs even if there is no wound on the second leg.
7. Give the patient the rest of the tube for daily re-application.
8. Off load the wound using best practices, etc.
9. Repeat Steps 2 through 9 at each weekly visit until the wound is resolved.

### Observations

1. The lidocaine will reduce pain in wounds like venous ulcers.
2. There will be almost no visible exudate after the initial treatment
   a. The fish oil is naturally anti-inflammatory.
   b. The lymph system and the venous system will drain more easily.
      i. You will see less leg swelling and therefore reduced resistance to circulation.
      ii. The wound will “drain in” not “drain out”.
         1. The skin will be visibly pinker and warmer to the touch.
   c. The C_{8&10} fats (in the virgin coconut oil) are absorbed in the stratum corneum where they quickly become fatty acids. Free fatty acid drops the skin pH to 5.5
      i. MMPs and other inflammatory enzymes are inhibited by the low pH.
      ii. The acid mantle is strengthened by the low pH and the wound is less likely to become infected.
         1. There is also 0.5% Geogard 361 an effective, anhydrous, low pH, multi-purpose preservative which helps preserve the wound bed from infection.
         2. The visible slough (biofilm) at the next visit will be reduced. You will sharp debride less often.
         3. Less time per patient is required in preparing the wound for off-loading.
      iii. The drop in pH will cause the red blood cells to drop off more oxygen at the wound site (Bohr Effect).
         1. This has the functional effect of increasing circulation even though blood flow may not have actually changed (liters/day).
3. The paste and the crème together will coat the wound margin with a layer of occlusive fat (mostly non-greasy C₁₆ from the palm oil). This helps protect the periwound margin from maceration.
   a. You will observe more rapid epithelialization because the margin is healthy.
4. The collagen paste preferably includes ascorbic acid (Vitamin C), copper gluconate and zinc monomethionine. These compounds help the collagen cross link and form structure as they are absorbed into the wound.
   a. New granulation will look firm and healthy (collagen+cross linking−excess moisture).
5. The paste preferably also has key nutrients (Vitamins A, D, E, K, lycopene, grape seed extract, aloe, MSM, curcumin plus calendula powder).
6. The Foot & Leg Crème has a complete nutrient package plus the same oil system as in the paste. The crème is rapidly absorbed.
   a. Observe:
      i. Exfoliation of dead skin
      ii. New pink skin
      iii. Closer pore structure after several weeks
      iv. Reduced swelling (use it up the leg until the vascular diameters are large (for maximum drainage).

### Normal Skin Appearance

Lidocaine USP mixtures with red palm oil, fish oil are also provided. The lidocaine is important because the increased circulation from the anti-inflammatory fish oil can increase pressure on previously under-used capillaries, causing pain in about 10% of patients. [Lidocaine HCl is not soluble in anhydrous oil mixtures.] Mixtures of red palm oil, fish oil and collagen as a paste and collagen as a sheet are also provided. The method of treating the periwound margin and the periwound with a red palm oil and fish oil emulsion was not previously disclosed. Collagen is an optional ingredient.

The fish oil crème shown in Table I is well received by patients because their skin is rejuvenated. Patients with lower extremity ulcers usually have vascular compromises of various kinds.

### Example 22

Miss C is a 35 year old woman with diabetic foot ulcer who is missing two toes. She is a person who takes pride in her appearance and is embarrassed by the foot deformity. After 6 weeks of treatment with the crème of Table I, the lower leg skin was unblemished and self-described as being soft to the touch. Her foot ulcer healed in 12 weeks.

### Analysis of Example 22

Miss C was so pleased with how her leg skin was rejuvenating that she was compliant with her doctor's orders. The ulcer healed rapidly.

While a number of exemplary aspects and embodiments have been discussed above, those of skill in the art will recognize certain modifications, permeations and additions and subcombinations thereof. It is therefore intended that the following appended claims and claims hereinafter introduced are interpreted to include all such modifications, permeations, additions and subcombinations that are within their true spirit and scope.

TABLE I
The Foot & Leg Crème
% by weight

| | |
|---|---|
| 0.470 | Allantoin |
| 0.210 | Aloe Vera |
| 0.025 | Arginine |
| 0.015 | Ascorbyl Palmitate |
| 0.450 | Beeswax |
| 0.215 | Benzethonium Chloride |
| 0.005 | Berberine Hydrochloride |
| 0.041 | Borage Oil |
| 0.015 | Boswellin |
| 0.050 | Carbopol |
| 3.627 | Cetyl Alcohol |
| 0.025 | Cetyl Lactate |
| 0.390 | Collagen |
| 0.020 | Colostrum |
| 0.002 | Copper Gluconate |
| 0.010 | DHEA |
| 2.559 | Distearyldimonium chloride |
| 0.033 | d-Pantothenyl alcohol |
| 0.075 | EDTA |
| 0.005 | Ferulic Acid |
| 3.780 | Glycerine |
| 4.070 | Glycerol Monolaurate |
| 0.010 | Grape Seed Extract |
| 0.150 | Jojoba Oil |
| 0.020 | Lavender Oil |
| 0.240 | Lemon Oil |
| 0.041 | Linoleic Acid |
| 0.005 | Lipoic Acid |
| 1.076 | Methyl Sulfonyl Methane |
| 0.500 | Mineral Oil |
| 0.030 | Neem Seed Oil |
| 0.048 | Omega 3 Fatty Acid |
| 1.758 | Omega-3 Fish Oil |
| 1.156 | Organic Coconut Oil |
| 1.734 | Palm Oil |
| 0.300 | Palm Tocotrienol |
| 0.011 | Phytantriol |
| 0.008 | Piperine |
| 0.130 | Polyoxyethylene 20 Sorbitan Monooleate |
| 0.020 | Pregnenolone |
| 0.500 | Preservative (Benzethonium chloride, benzyl alcohol, |
| | phenoxyethanol, benzoic acid, dehydro acetic acid, |
| | salicylic acid) |
| 0.010 | Proline |
| 1.950 | Propylene Glycol |
| 0.020 | Pyrrolidone Carboxylic Acid |
| 0.375 | Sclerotium gum |
| 0.200 | Shark Liver Oil |
| 0.352 | Shea Nut Oil |
| 0.010 | Silica Gel |
| 1.238 | Stearic Acid |
| 1.826 | Triethanolamine |
| 0.005 | Vitamin A |
| 0.300 | Vitamin C |
| 0.005 | Vitamin D |
| 0.150 | Vitamin E |
| 0.005 | Vitamin K |
| 61.403 | Water |
| 0.008 | Zinc Monomethionine |
| 0.020 | Zinc Sulfate |

TABLE 2
VITAL CHOICE COMPOSITE SOCKEYE SALMON OIL ANALYSIS:
Fatty Acid Profile

| Component Name | Carbon Chain | Pct | Mg | Om-3* | Om-6 | Om-7 | Om-9 | Other |
|---|---|---|---|---|---|---|---|---|
| Myristic Acid | C14:0 | 4.9 | 49 | | | | | 4.9 |
| Trans Myristelaidic Acid | C14:1 | 0.3 | 3 | | | | | 0.3 |
| Myristoleic Acid | C14:1w5 | 0.2 | 2 | | | | | 0.2 |
| Pentadecanoic Acid | C15:0 | 0.6 | 6 | | | | | 0.6 |
| Pentadecenoic Acid | C15:1 | 0.1 | 1 | | | | | 0.1 |
| Palmitic Acid¹ | C16:0 | 18.5 | 185 | | | | | 18.5 |
| Trans Palmitelaidic Acid | C16:1t | 0.8 | 8 | | | | | 0.8 |
| Palmitoleic Acid | C16:1w7 | 5.4 | 54 | | | 5.4 | | |
| Hexadecadienoic Acid | C16:2 | 0.6 | 6 | | | | | 0.6 |
| Hexadecatrienoic Acid | C16:3 | 0.3 | 3 | | | | | 0.3 |
| Hexadecatetraenoic Acid | C16:4 | 0.9 | 9 | | | | | 0.9 |
| Heptadecanoic Acid | C17:0 | 0.7 | 7 | | | | | 0.7 |
| Heptadecenoic Acid | C17:1w7 | 0.2 | 2 | | | 0.2 | | |
| Stearic Acid | C18:0 | 2.4 | 24 | | | | | 2.4 |
| Oleic Acid² | C18:1w9 | 22.9 | 229 | | | | 22.9 | |
| Trans Elaidic Acid | C18:1t | 0.2 | 2 | | | | | 0.2 |
| Linoleic Acid | C18:2w6 | 1.5 | 15 | | 1.5 | | | |
| Trans Linolelaidic Acid | C18:2t | 1.5 | 15 | | | | | 1.5 |
| Gamma-Linolenic Acid | C18:3w6 | 0.2 | 2 | | 0.2 | | | |
| Linolenic Acid | C18:3w3 | 0.9 | 9 | 0.9 | | | | |
| Octadecatetraenoic Acid | C18:4w3 | 5.9 | 59 | 5.9 | | | | |
| Arachidic Acid | C20:0 | 0.1 | 1 | | | | | 0.1 |
| Eicosenoic Acid | C20:1w9 | 0.1 | 1 | | | | 0.1 | |
| Eicosadienoic Acid | C20:2w6 | 2.5 | 25 | | 2.5 | | | |
| Eicosatrienoic Acid | C20:3w3 | 1.9 | 19 | 1.9 | | | | |
| Hommogamma Linoleic Acid | C20:3w6 | 1.3 | 13 | | 1.3 | | | |
| Arachidonic Acid³ | C20:4w6 | 4.8 | 48 | | 4.8 | | | |
| Eicosapentaenoic Acid (EPA) | C20:5w3 | 8.9 | 89 | 8.9 | | | | |
| Behenic Acid | C22:0 | 0.7 | 7 | | | | | 0.7 |
| Erucic Acid | C22:1w9 | 0.3 | 3 | | | | 0.3 | |
| Docosanoic Acid | C22:1w11 | 1.3 | 13 | | | | | 1.3 |
| Docosapentaenoic Acid (DPA) | C22:5w3 | 1.8 | 18 | 1.8 | | | | |
| Docosahexaenoic Acid (DHA) | C22:6w3 | 7.0 | 70 | 7.0 | | | | |
| Nervonic Acid | C24:1w9 | 0.3 | 3 | | | | 0.3 | |
| Total | | 100.00 | 1000 | 26.40 | 10.30 | 5.60 | 23.60 | 34.10 |

*Omega totals are percentages
¹Palmitic acid is a major fatty acid present in meat and dairy products
²Oleic acid is the major fatty acid in olive and canola oils
³Arachidonic acid is an essential fatty acid used to synthesize regulatory molecules such as prostaglandins and thromboxanes; a component of cell membranes released on their injury

1. A cosmeceutically acceptable anhydrous formulation comprising:
red palm oil;
a triglyceride;
wherein the ratio of C_{>18} to C_{≦18} of polyunsaturated fatty acids is greater than 1.

2. A cosmeceutically acceptable anhydrous formulation as set forth in claim 1, further comprising:
fish collagen.

3. A cosmeceutically acceptable anhydrous topical formulation comprising:
red palm oil;
fish oil;
said formulation being a homogenous liquid at room temperature (˜72° F.).

4. A cosmeceutically acceptable anhydrous topical formulation as set forth in claim 3, further comprising:
coconut oil;
shea butter.

5. A cosmeceutically acceptable anhydrous topical formulation as set forth in claim 4, wherein:
the mole ratio of C₁₆ substantially equals C_{18.1};
the sum of C₄₋₁₀ and monounsaturated fats divided by the sum of polyunsaturated fats substantially equals 2.

6. A cosmeceutically acceptable anhydrous topical formulation as set forth in claim 3, further comprising:
an acidic preservative which strengthens the effectiveness of the acid mantle of the skin.

7. A cosmeceutically acceptable anhydrous topical formulation as set forth in claim 3, further comprising:
a winterized cationic surfactant which reduces malodor.

8. A cosmeceutically acceptable anhydrous topical formulation as set forth in claim 7, wherein:
said cationic surfactant is benzethonium chloride.

9. A cosmeceutically acceptable anhydrous topical formulation as set forth in claim 3, wherein:
said red palm oil and said fish oil are present in a weight ratio of about 1:1.

10. A cosmeceutically acceptable anhydrous topical formulation as set forth in claim 3, further comprising:
fish collagen.

11. A cosmeceutically acceptable anhydrous formulation comprising:
red palm oil;
very long chain omega 3 and 6 fatty acids greater than C₁₈.

12. A cosmeceutically acceptable anhydrous formulation as set forth in claim 11, further comprising:
fish collagen.

13. A cosmeceutically acceptable aqueous emulsion formulation comprising:
red palm oil;
a triglyceride;
wherein the ratio of C_{>18} to C_{≦18} of polyunsaturated fatty acids is greater than 1.

14. A cosmeceutically acceptable aqueous emulsion formulation as set forth in claim 13, further comprising:
fish collagen.

15. A cosmeceutically acceptable aqueous emulsion topical formulation comprising:
red palm oil;
fish oil;
said formulation being a homogenous liquid at room temperature (˜72° F.).

16. A cosmeceutically acceptable aqueous emulsion topical formulation as set forth in claim 15, further comprising:
coconut oil;
shea butter.

17. A cosmeceutically acceptable aqueous emulsion topical formulation as set forth in claim 16, wherein:
the mole ratio of C₁₆ substantially equals C_{18.1};
the sum of C₄₋₁₀ and monounsaturated fats divided by the sum of polyunsaturated fats substantially equals 2.

18. A cosmeceutically acceptable anhydrous topical formulation as set forth in claim 15, further comprising:
an acidic preservative which strengthens the effectiveness of the acid mantle of the skin.

19. A cosmeceutically acceptable aqueous emulsion formulation comprising:
red palm oil;
very long chain omega 3 and 6 fatty acids greater than C₁₈.

20. A cosmeceutically acceptable aqueous emulsion formulation as set forth in claim 11, further comprising:
fish collagen.
